(19) United States
(12) Patent Application Publication
(10) Pub. No.: US 2003/0228365 A1
(43) Pub. Date: Dec. 11, 2003

(54) Pharmaceutical formulation

(76) Inventors: Fortuna Haviv, Deerfield, IL (US); Jack Henkin, Highland Park, IL (US); Luk C. Li, Lake Forest, IL (US); Fanfeng Ma, Vernon Hills, IL (US); Yi Shi, Libertyville, IL (US); Jing Feng Song, Waukegan, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/165,143

(22) Filed: Jun. 7, 2002

(51) Int. Cl. A61K 9/14; A61K 38/10; A61K 38/08

(52) U.S. Cl. 424/486; 514/16

(57) ABSTRACT

Parenteral formulations of peptides which are useful for sustained release are disclosed. Also disclosed are methods of preparation for the formulations.

### TECHNICAL FIELD

The present invention relates to parenteral formulations of peptides. These formulations are useful for sustained release of the peptides. Methods for the preparation of the formulations and methods for their use are also disclosed.

### BACKGROUND OF THE INVENTION

The peptides of the present invention have been shown to inhibit angiogenesis, the fundamental process by which new blood vessels are formed that is essential to a variety of normal body activities (such as reproduction, development, and wound repair). Although angiogenesis is a highly regulated process under normal conditions, many diseases (characterized as “angiogenic diseases”) are driven by persistent unregulated angiogenesis. Otherwise stated, unregulated angiogenesis may either cause a particular disease directly or exacerbate an existing pathological condition.

In many instances, the therapeutic effectiveness of a pharmaceutically active peptide depends on its continued presence in vivo over prolonged time periods. A sustained release formulation or sustained drug delivery is desirable to avoid the need for repeated administrations. For example, luteinizing hormone releasing hormone (LHRH) superagonists, such as leuprolide, typically are encapsulated within a polymeric membrane to produce microparticles suitable for depot injection that provide sustained delivery of the superagonist over several weeks to months. However, additional sustained delivery formulations for administering pharmaceutically active peptides in vivo continuously for prolonged time periods are still needed.

### SUMMARY OF THE INVENTION

In one embodiment, the present invention provides a pharmaceutical composition comprising:

(a) a therapeutically effective amount of a compound of formula (I) R¹-Xaa₁-Xaa₂-Xaa₃-Xaa₄-Xaa₅-Xaa₆-Ile-Arg-Pro-Xaa₁₀ (I), (SEQ ID NO: 1);

wherein

R¹ is CH₃—C(O)—;

Xaa₁ is absent or sarcosyl;

Xaa₂ is absent or glycyl;

Xaa₃ is absent or selected from the group consisting of glutaminyl and valyl;

Xaa₄ is absent or selected from the group consisting of D-alloisoleucyl and D-isoleucyl;

Xaa₅ is selected from the group consisting of seryl and threonyl;

Xaa₆ is selected from the group consisting of glutaminyl, norvalyl, and seryl; and

Xaa₁₀ is selected from the group consisting of —NHCH₂CH₃ and D-alanylethylamide; provided that when Xaa₄ is D-alloisoleucyl, Xaa₁ is absent;

(b) poly(lactide-co-glycolide); and

(c) an organic solvent.

In a preferred embodiment, the compound of formula (I) is selected from the group consisting of

N-Ac-Sar-Gly-Val-DIle-Thr-Nva-Ile-Arg-ProNHCH₂CH₃;

N-Ac-Sar-Gly-Val-DIle-Thr-Gln-Ile-Arg-ProNHCH₂CH₃;

N-Ac-DalloIle-Thr-Ser-Ile-Arg-ProNHCH₂CH₃;

N-Ac-Thr-Gln-Ile-Arg-ProNHCH₂CH₃ (SEQ ID NO:2);

N-Ac-DalloIle-Ser-Ser-Ile-Arg-ProNHCH₂CH₃;

N-Ac-Gly-Val-DalloIle-Ser-Gln-Ile-Arg-ProNHCH₂CH₃; and

N-Ac-Gly-Gln-DIle-Thr-Nva-Ile-Arg-Pro-DAlaNH₂.

In another preferred embodiment, the pharmaceutical composition comprises between about 1% and about 15% (w/w) of the compound of formula (I). More preferably, the pharmaceutical composition comprises between about 3% and about 5% (w/w) of the compound of formula (I).

In another preferred embodiment, the pharmaceutical composition comprises between about 25% and about 45% (w/w) poly(lactide-co-glycolide). More preferably, the pharmaceutical composition comprises about 35% (w/w) poly(lactide-co-glycolide).

In another preferred embodiment, the poly(lactide-co-glycolide) of the pharmaceutical composition has a weight of between about 6 and about 60 KD. More preferably, the weight is between about 13 and about 24 KD.

In another preferred embodiment, the organic solvent used in the pharmaceutical composition is selected from the group consisting of N-methyl-2-pyrrolidinone, triacetin, and mixtures thereof. More preferably, the organic solvent is a mixture of N-methyl-2-pyrrolidinone and triacetin in a weight ratio of between about 1:2 to about 6:1. Most preferably, the N-methyl-2-pyrrolidinone and triacetin are in a weight ratio of about 2:1 or about 1:1.

In a most preferred embodiment, the present invention provides a pharmaceutical composition comprising

(a) about 3% to about 5% (w/w) of the compound of formula (Ia) N-Ac-Sar-Gly-Val-DIle-Thr-Nva-Ile-Arg-ProNHCH₂CH₃ (Ia);

(b) about 35% (w/w) poly(lactide-co-glycolide); and

(c) about a 2:1 (w/w) mixture of N-methylpyrrolidinone and triacetin.

In another most preferred embodiment, the present invention provides a pharmaceutical composition comprising

(a) about 3% (w/w) of the compound of formula (Ib) N-Ac-Sar-Gly-Val-DIle-Thr-Gln-Ile-Arg-ProNHCH₂CH₃ (Ib);

(b) about 35% (w/w) poly(lactide-co-glycolide); and

(c) about a 1:1 (w/w) mixture of N-methylpyrrolidinone and triacetin.

In another embodiment, the present invention provides a method for preparing a pharmaceutical composition comprising:

(a) combining between about 25% and about 45% (w/w) poly(lactide-co-glycolide) and about 1% to about 15% (w/w) of a compound of formula (I) in an organic solvent; and

(b) stirring the product of step (a), preferably at a temperature of between about 20° C. and about 25° C.

In another embodiment, the present invention provides a method for preparing a pharmaceutical composition comprising:

(a) dissolving between about 25% and about 45% (w/w) poly(lactide-co-glycolide) in an organic solvent selected from the group consisting of N-methyl-2-pyrrolidinone, triacetin, 2-pyrrolidinone, and mixtures thereof;

(b) treating the product of step (a) with about 2% to about 10% (w/w) of a compound of formula (I); and

(c) stirring the product of step (b).

Preferably, the compound of formula (I) is selected from the group consisting of

N-Ac-Sar-Gly-Val-DIle-Thr-Nva-Ile-Arg-ProNHCH₂CH₃;

N-Ac-Sar-Gly-Val-DIle-Thr-Gln-Ile-Arg-ProNHCH₂CH₃;

N-Ac-DalloIle-Thr-Ser-Ile-Arg-ProNHCH₂CH₃;

N-Ac-Thr-Gln-Ile-Arg-ProNHCH₂CH₃ (SEQ ID NO:2);

N-Ac-DalloIle-Ser-Ser-Ile-Arg-ProNHCH₂CH₃;

N-Ac-Gly-Val-DalloIle-Ser-Gln-Ile-Arg-ProNHCH₂CH₃; and

N-Ac-Gly-Gln-DIle-Thr-Nva-Ile-Arg-Pro-DAlaNH₂.

In a preferred embodiment, the pharmaceutical composition comprises about 35% (w/w) poly(lactide-co-glycolide).

In another preferred embodiment, the poly(lactide-co-glycolide) has a weight of between about 13 and about 24 KD.

In another preferred embodiment, the organic solvent used in the pharmaceutical composition is selected from the group consisting of N-methyl-2-pyrrolidinone, triacetin, and mixtures thereof. More preferably, the organic solvent is a mixture of N-methyl-2-pyrrolidinone and the triacetin in a weight ratio of from about 1:2 to about 6:1. Most preferably, the N-methyl-2-pyrrolidinone and the triacetin are in a weight ratio of from about 2:1 to about 1:1.

In a most preferred embodiment, the present invention provides a method for preparing a pharmaceutical composition comprising:

(a) dissolving about 35% (w/w) 13 KD poly(lactide-co-glycolide) in about a 2:1 (w/w) mixture of N-methyl-2-pyrrolidinone and triacetin;

(b) treating the product of step (a) with about 3% to about 5% (w/w) of the compound of formula (Ia); and

(c) stirring the product of step (b) at about 20° C. to about 25° C.

In another most preferred embodiment, the present invention provides a method for preparing a pharmaceutical composition comprising:

(a) dissolving about 35% (w/w) 13 KD poly(lactide-co-glycolide) in about a 1:1 (w/w) mixture of N-methyl-2-pyrrolidinone and triacetin;

(b) treating the product of step (a) with about 3% (w/w) of the compound of formula (Ib); and

(c) stirring the product of step (b) at about 20° C. to about 25° C.

In another embodiment, the present invention provides a method for providing sustained delivery of a peptide comprising administering to a subject a pharmaceutical composition comprising

(a) about 1% to about 15% (w/w) of a compound of formula (I) R¹-Xaa₁-Xaa₂-Xaa₃-Xaa₄-Xaa₅-Xaa6-Ile-Arg-Pro-Xaa₁₀ (I), (SEQ ID NO: 1);

wherein

R¹ is CH₃—C(O)—;

Xaa₁ is absent or sarcosyl;

Xaa₂ is absent or glycyl;

Xaa₃ is absent or selected from the group consisting of glutaminyl and valyl;

Xaa₄ is absent or selected from the group consisting of D-alloisoleucyl and D-isoleucyl;

Xaa₅ is selected from the group consisting of seryl and threonyl;

Xaa₆ is selected from the group consisting of glutaminyl, norvalyl, and seryl; and

Xaa₁₀ is selected from the group consisting of —NHCH₂CH₃ and D-alanylethylamide; provided that when Xaa₄ is D-alloisoleucyl, Xaa₁ is absent;

(b) about 25% to about 45% (w/w) poly(lactide-co-glycolide); and

(c) an organic solvent selected from the group consisting of N-methyl-2-pyrrolidinone, triacetin, and mixtures thereof.

In a preferred embodiment, the compound of formula (I) is selected from the group consisting of

N-Ac-Sar-Gly-Val-DIle-Thr-Nva-Ile-Arg-ProNHCH₂CH₃;

N-Ac-Sar-Gly-Val-DIle-Thr-Gln-Ile-Arg-ProNHCH₂CH₃;

N-Ac-DalloIle-Thr-Ser-Ile-Arg-ProNHCH₂CH₃;

N-Ac-Thr-Gln-Ile-Arg-ProNHCH₂CH₃ (SEQ ID NO:2);

N-Ac-DalloIle-Ser-Ser-Ile-Arg-ProNHCH₂CH₃;

N-Ac-Gly-Val-DalloIle-Ser-Gln-Ile-Arg-ProNHCH₂CH_{3;} and

N-Ac-Gly-Gln-DIle-Thr-Nva-Ile-Arg-Pro-DAlaNH₂.

### BRIEF DESCRIPTION OF THE DRAWINGS

FIG. 1 illustrates the in vitro release profile of the compound of formula (Ia) from PLG (13 KD) gel formulations at 37° C.

FIG. 2 illustrates the mean plasma concentrations of the compound of formula (Ia) in dogs following single subcutaneous injections of PLG (13 KD) gel formulations.

FIG. 3 illustrates the in vitro drug release profiles of the compound of formula (Ib) from PLG (13 KD) gel formulations at 37° C.

FIG. 4 illustrates the mean plasma concentrations of the compound of formula (Ib) in dogs following single subcutaneous injections of PLG (13 KD) gel formulations.

FIG. 5 illustrates the mean plasma concentrations of the compound of formula (Ib) in monkeys following single subcutaneous injections of PLG (13 KD) gel formulations.

FIG. 6 illustrates the in vitro drug release profiles of the compound of formula (Ia) from PLG (24 KD) gel formulations at 37° C.

### DETAILED DESCRIPTION OF THE INVENTION

The present invention relates to sustained release formulations of peptides that contain poly(lactide-co-glycolide) and organic solvents. These formulations have demonstrated in vitro as well as in vivo activity.

As used in the present specification the following terms have the meanings indicated:

As used herein, the singular forms “a”, “an”, and “the” include plural reference unless the context clearly dictates otherwise.

The term “organic solvent,” as used herein, refers to a single organic solvent or a mixture of two or more organic solvents that demonstrates no undue toxicity when added to the formulations of the present invention. Preferred organic solvents of the present invention include N-methyl-2-pyrrolidinone, 2-pyrrolidinone, triacetin, dimethylsulfoxide, benzyl benzoate, and mixtures thereof.

The term “sustained delivery,” as used herein, refers to the continual delivery of a pharmaceutical agent in vivo over a period of time following administration, preferably at least several days, a week, or several weeks. Sustained delivery of the agent can be demonstrated by, for example, the continued therapeutic effect of the agent over time. Alternatively, sustained delivery of the agent may be demonstrated by detecting the presence of the agent in vivo over time.

The pharmaceutical formulation contains a therapeutically effective amount of the compound of formula (I). The term “therapeutically effective amount,” as used herein, refers to an amount effective, at dosages and for periods of time necessary, to achieve the desired result. A therapeutically effective amount of the compound of formula (I) may vary according to factors such as the disease state, age, and weight of the individual, and the ability of the compound (alone or in combination with one or more other drugs) to elicit a desired response in the individual. Dosage regimens may be adjusted to provide the optimum therapeutic response. A therapeutically effective amount is also one which any toxic or ads detrimental effects of the compound are outweighted by the therapeutically beneficial effects. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are only exemplary and are not intended to limit the scope or practice of the claimed composition.

Many diseases (characterized as “angiogenic diseases”) are driven by persistent unregulated angiogenesis. For example, ocular neovascularization has been implicated as the most common cause of blindness. In certain existing conditions such as arthritis, newly formed capillary blood vessels invade the joints and destroy cartilage. In diabetes, new capillaries formed in the retina invade the vitreous, bleed, and cause blindness. For example, ocular neovascularization has been implicated as the most common cause of blindness. In certain existing conditions such as arthritis, newly formed capillary blood vessels invade the joints and destroy cartilage. In diabetes, new capillaries formed in the retina invade the vitreous, bleed, and cause blindness. Growth and metastasis of solid tumors are also angiogenesis-dependent (Folkman, J., Cancer Res., 46: 467-473 (1986), Folkman, J., J. Natl. Cancer Inst., 82: 4-6 (1989)). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites, such as the liver, the lung, and the bones (Weidner, N., et. al., N. Engl. J. Med., 324(1): 1-8 (1991)).

The compounds of the invention, including not limited to those specified in the examples, possess antiangiogenic activity. As angiogenesis inhibitors, such compounds are useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compounds may also be useful in treating solid tumors arising from hematopoietic malignancies such as leukemias (i.e., chloromas, plasmacytomas and the plaques and tumors of mycosis fungosides and cutaneous T-cell lymphoma/leukemia) as well as in the treatment of lymphomas (both Hodgkin's and non-Hodgkin's lymphomas). In addition, these compounds may be useful in the prevention of metastases from the tumors described above either when used alone or in combination with radiotherapy and/or other chemotherapeutic agents. The compounds of the invention can also be useful in the treatment of the aforementioned conditions by mechanisms other than the inhibition of angiogenesis.

Further uses include the treatment and prophylaxis of autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemagiomas, and capillary proliferation within atherosclerotic plaques; Osler-Webber Syndrome; myocardial angiogenesis; plaque neovascularization; telangiectasia; hemophiliac joints; angiofibroma; and wound granulation. Other uses include the treatment of diseases characterized by excessive or abnormal stimulation of endothelial cells, including not limited to intestinal adhesions, Crohn's disease, atherosclerosis, scleroderma, and hypertrophic scars, i.e., keloids. Another use is as a birth control agent, by inhibiting ovulation and establishment of the placenta. The compounds of the invention are also useful in the treatment of diseases that have angiogenesis as a pathologic consequence such as cat scratch disease (Rochele minutesalia quintosa) and ulcers (Helicobacter pylori). The compounds of the invention are also useful to reduce bleeding by administration prior to surgery, especially for the treatment of resectable tumors.

Unless indicated otherwise by a “D” prefix, e.g., DAla or DIle, the stereochemistry of the α-carbon of the amino acids and aminoacyl residues in peptides described in this specification and the appended claims is the natural or “L” configuration.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature. To the extent that the names and abbreviations of amino acids and aminoacyl residues employed in this specification and appended claims differ from those suggestions, they will be made clear to the reader. Some abbreviations useful in describing the invention are defined below in the following Table 1.

TABLE 1
Amino Acid Abbreviations

| Abbreviation | Definition |
|---|---|
| DAlaNH₂ | D-alanylamide |
| DalloIle | D-alloisoleucyl |
| N-Ac-DalloIle | N-acetyl-D-alloisoleucyl |
| Arg | arginyl |
| Gln | glutaminyl |
| Gly | glycyl |
| N-Ac-Gly | N-acetylglycyl |
| Ile | isoleucyl |
| DIle | D-isoleucyl |
| Nva | norvalyl |
| Pro | prolyl |
| ProNHCH₂CH₃ | prolyl-N-ethylamide |
| N-Ac-Sar | N-acetylsarcosyl |
| Ser | seryl |
| Thr | threonyl |
| N-Ac-Thr | N-acetylthreonyl |
| Val | valyl |

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects. The contents of all references, patents, and published patent applications cited throughout this application are hereby incorporated by reference.

Poly(lactide-co-glycolide) (PLG) was purchased from Alkermes, Inc. The ratio of the two monomers (PL:PG) was 50:50 or 75:25. 1-Methyl-2-pyrrolinone (NMP) was purchased from ISP technologies and triacetin (glycerol triacetate) (TA) was purchased from Aldrich.

### EXAMPLE 1

### Preparation of the Formulations of Compound of Formula (Ia) in PLG (13 KD) Gels

(a) Formulation of 5% Compound of Formula (Ia) in 35% PLG Gel and NMP/TA (2:1)

(Formulation I)

A mixture of NMP and TA (2:1, w/w) was prepared using of 14.993 grams of TA and 30.022 grams of NMP. A portion of this solvent mixture (19.505 g) was stirred at room temperature with 10.515 g of PLG (13 KD, 50:50 polymer ratio). The resulting PLG (35%) solution was transparent and viscous. A portion of the PLG solution (12.026 g) was treated with the previously lyophilized compound of formula (Ia) (prepared according to the procedure described in WO99/61476, 631 mg). The mixture was stirred at room temperature until a clear gel formed. The resulting PLG formulation (formulation I) consisted of 4.98% compound of formula (Ia), 33.28% PLG, 41.18% NMP and 20.56% TA (w/w), and could be stored under refrigeration.

(b) Formulation of 5% Compound of Formula (Ia) in 30% PLG Gel and NMP/TA (2:1)

(Formulation II)

A 30% PLG solution in NMP/TA (2:1, w/w) was prepared from 9.018 g of the 35% PLG solution in NMP/TA made in (a) and 1.502 g of a 2:1 NMP/TA solvent mixture. The resulting 30% PLG solution (9.008 g) was treated with 473.5 mg of the compound of formula (Ia) and stirred at room temperature resulting in a viscous liquid formulation (formulation II) which consisted of 4.99% compound of formula (Ia), 28.53% PLG, 44.34% NMP and 22.14% TA (w/w).

(c) Formulation of 5% Compound of Formula (Ia) in 25% PLG Gel and NMP/TA (2:1)

(Formulation III)

A mixture of 35% PLG solution in NMP/TA (2:1) made in (a) (7.4999 g) was diluted with 3.0140 g of a solvent mixture of NMP/TA (2:1). A portion of this solution (9.008 g) was stirred with 471.5 mg of the compound of formula (Ia) to provide a formulation (formulation III) which consisted of 5.01% compound of formula (Ia), 23.73% PLG, 47.53% NMP and 23.73% TA (w/w).

(d) Formulation of 8% Compound of Formula (Ia) in 35% PLG Gel and NMP/TA (2:1)

A gel formulation of 8% compound of formula (Ia) was prepared from 0.1747 g of the compound of formula (Ia) and 2.0638 g of 35% PLG solution in NMP/TA (2:1) (prepared in (a)). The mixture was stirred at room temperature to provide a liquid formulation which consisted of 7.80% compound of formula (Ia), 32.25% PLG, 39.96% NMP and 19.99% TA (w/w).

(e) Formulation of 5% Compound of Formula (Ia) in PLG Gel and NMP/TA (1:1)

A mixture of 1.015 g of 35% PLG (13 KD, 50:50 polymer ratio) solution in NMP was mixed with 1.0016 g of 35% PLG (13 KD, 50:50 polymer ratio) solution in TA. A portion of the resulting solution (1.0046 g) was stirred with 50.8 mg of the compound of formula (Ia) at room temperature to provide a clear formulation which consisted of 4.81% compound of formula (Ia), 33.26% PLG, 31.16% NMP and 30.77% TA (w/w).

### EXAMPLE 2

### Potency Determination

A sample of the compound of formula (Ia) in PLG gel was dissolved in aqueous acetonitrile and further diluted with water. The precipitated polymer was subsequently removed by filtration through a membrane filter. The concentration of the compound of formula (Ia) in the filtrate was determined by HPLC. The compound of formula (Ia) could be completely recovered from the PLG gel. There was no extensive degradation of found by HPLC.

### EXAMPLE 3

### In Vitro Release of Compound of Formula (Ia) from PLG Gels

The samples of gel formulations of the compound of formula (Ia) in PLG and NMP/TA were immersed in 5 mM PBS buffer (pH 7.4) and incubated at 37° C. At a predetermined time, 1 mL of the dissolution medium was withdrawn from the dissolution container, filtered, and assayed for the concentration of the compound of formula (Ia) by IHPLC. Fresh PBS buffer (1 mL) was added to replace the withdrawn medium.

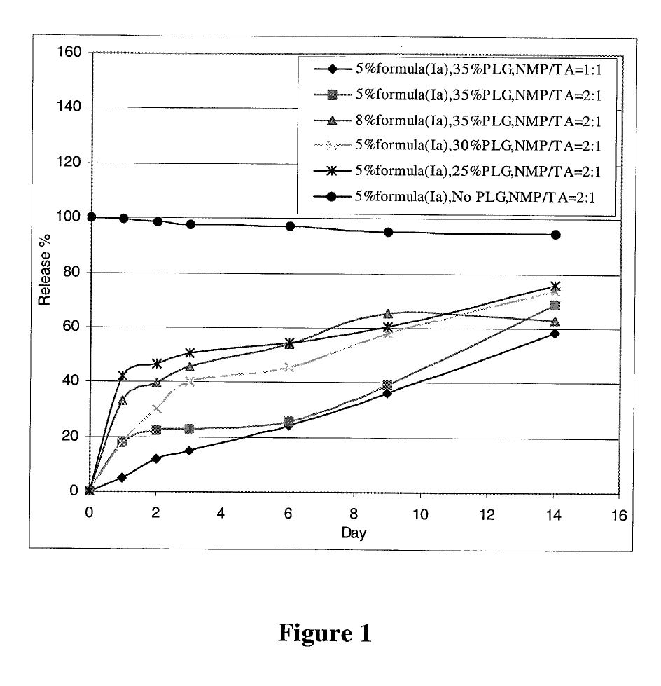

As shown in FIG. 1, a solution of 5% of the compound of formula (I) in 2:1 NMP/TA showd no sustained release. Alternatively, PLG gels containing 5% or 8% of the compound of formula (I); 25%, 30% or 35% PLG; and NMP/TA in either a 2:1 or 1:1 ratio showed a more gradual release.

### EXAMPLE 4

### Pharmacokinetics Studies of the Compound of Formula (Ia) in PLG Gels

In vivo pharmacokinetic studies of the compound of formula (Ia) in PLG gels were performed using dogs. Five groups of dogs were tested by subcutaneous injection. Three groups were given subcutaneous injections of the three gel formulations: formulations I, II, and III from Example 1. Each of the formulations was administered at a dose of 50 mg/dog. One control group was given a subcutaneous injection of the compound of formula (I) in 5% dextrose in water (D5W) at a dose of 50 mg/dog and another control group was administrated placebos consisting of 30% PLG in a solvent mixture of NMP and TA (2:1). Nine blood samples were taken from the dogs during the first 24 hours after dosing, followed by daily sampling for 14 days. No irritation was seen at the injection site in any of the dogs that were given the PLG gels.

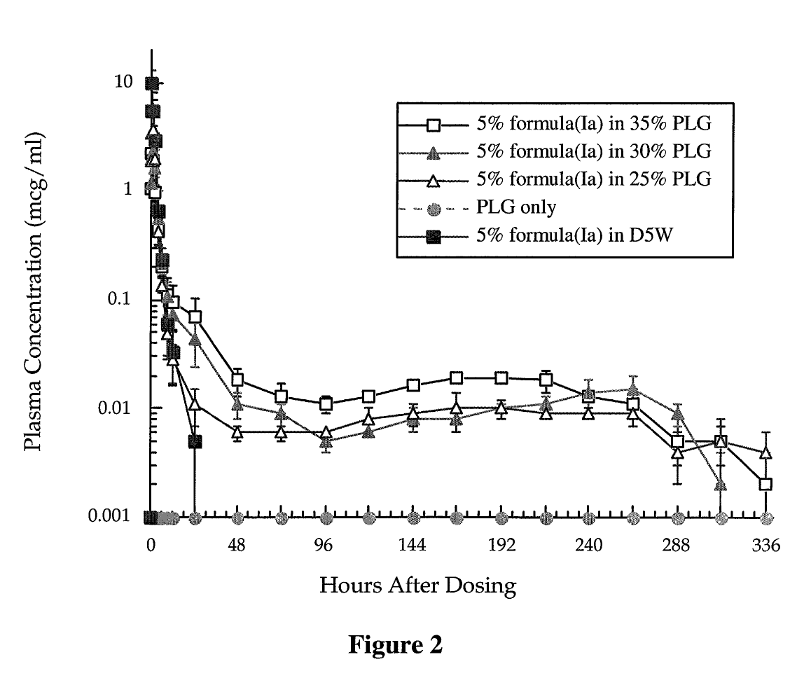

Concentrations of the compound of formula (Ia) in plasma were determined by HPLC-MS. The results are summarized in FIG. 2. The compound of formula (Ia) was rapidly absorbed from the injectable solution, with the peak concentration observed within one hour of dosing. A two-week sustained release of the compound of formula (Ia) was shown by all of the dogs injected with the gel formulations in 25-30% PLG and NMP/TA (2:1). Drug plasma concentrates were observable for all dogs up to 12 days after dosing and the concentrations were still detectable in ˜50% of the dogs by day 14. In comparison, the group that was given the compound of formula (Ia) in D5W yielded drug plasma concentrations below the limits of quantitation within 24 hours after dosing.

### EXAMPLE 5

### Preparation of the Formulations of the Compound of Formula (Ib) in PLG Gels

(a) Formulation of 3% Compound of Formula (Ib) in 35% PLG and NMP/TA (1:1)

(Formulation IV)

A 35% PLG solution in NMP/TA (1:1) was prepared by combining 8.140 g of TA, 8.132 g of NMP, and 8.761 g of PLG (13 KD, 50:50 polymer ratio). A portion of the mixture (4.414 g) was treated with of the compound of formula (Ib) (prepared according to the procedure described in WO99/61476, 136.1 mg) and stirred with a magnetic stirring bar at room temperature until a homogeneous gel was formed. The resulting -PLG gel (formulation IV) consisted of 2.99% compound of formula (Ib), 33.95% PLG, 31.53% NMP and 31.53% TA (w/w) and could be stored under refrigeration.

(b) Formulation of 3% Compound of Formula (Ib) in 35% PLG and NMP/TA (2:1)

(Formulation V)

A 35% PLG solution was prepared by combining 4.329 g of TA, 8.712 g of NMP, and 7.003 g of PLG (13 KD, 50:50 polymer ratio). A portion of the solution (4.844 g) was treated with 144.7 g of the compound of formula (Ib) and stirred at room temperature. The resulting PLG gel (formulation V) consisting of 2.90% compound of formula (Ib), 33.93% PLG, 42.11% NMP and 21.06% TA (w/w) was stored under refrigeration.

### EXAMPLE 6

### In Vitro Drug Release of Compound of Formula (Ib) from PLG (13 KD) Gels

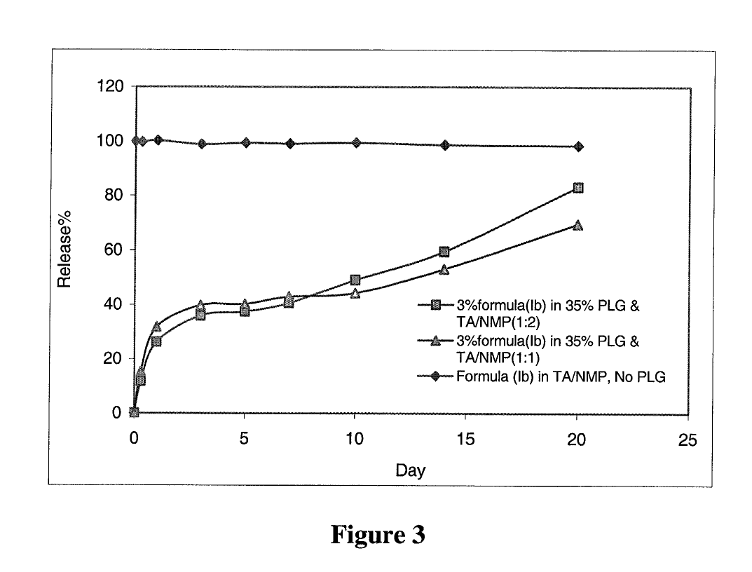

The in vitro drug release of the compound of formula (Ib) from the PLG gel formulations (IV) and (V) (from Example 5) was determined by the method described in Example 3. As shown in FIG. 3, both formulations exhibited in vitro sustained release for two weeks, as opposed to the control, which showed no sustained release.

### EXAMPLE 7

### Pharmacokinetics Studies of ABT-567 in PLG Gels

(a) Dog study

One in vivo pharmacokinetic study was done using dogs. Two groups of dogs were injected subcutaneously with the gel formulations IV and V (from Example 5), and a control group of dogs was injected with a solution of the compound of formula (Ib) in D5W. Each dog was administrated with a dose of 30 mg of formulation. The drug release was determined by the measurement of the concentration of the compound of formula (Ib) in plasma using the same procedure as described in Example 4.

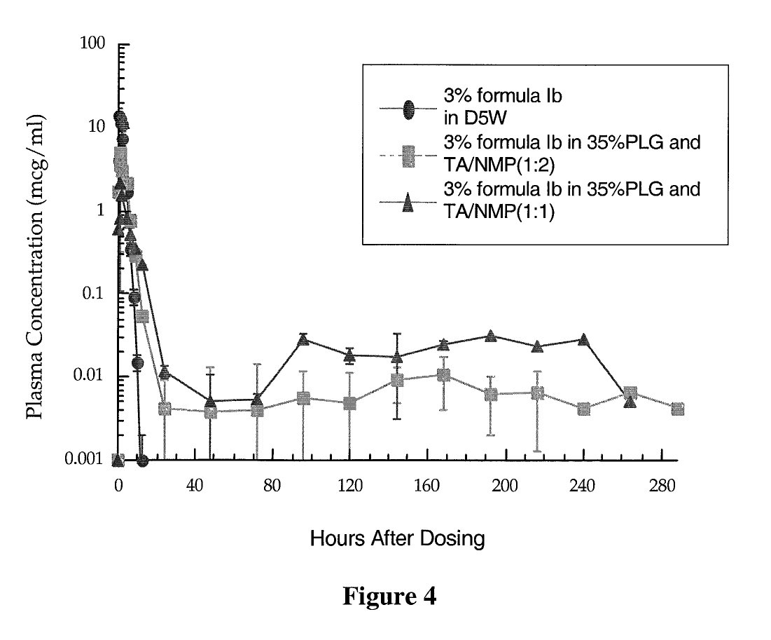

As shown in FIG. 4, sustained release was seen in all of the dogs injected with the formulations IV and V. All of the dogs dosed with formulations IV and V exhibited measurable drug plasma concentrations (above 10 ng/mL) up to 12 days after injection. In comparison, the group receiving the control in D5W yielded drug plasma concentrations below the limits of quantitation within 24 hours after dosing.

(b) Monkey study

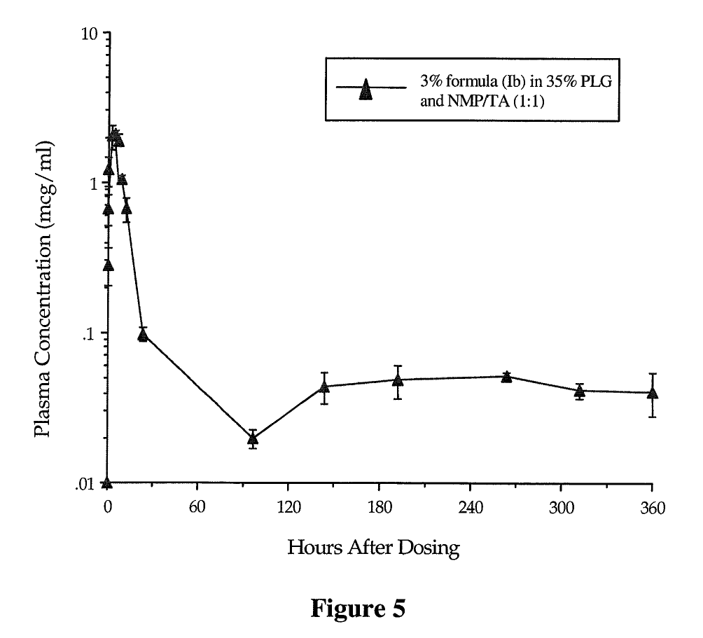

Another in vivo pharmacokinetic was performed using monkeys. Each monkey was injected subcutaneously with formulation IV (from Example 5) at a dose of 30 mg/monkey. Nine blood samples were obtained from the testing monkeys during the first 24 hours after dosing, with intermittent sampling for the following 15 days. The plasma concentrations of the compound of formula (Ib) were determined by HPLC-MS. As shown in FIG. 5, the release profile of formulation IV in monkeys was similar to that described in the dog study (a). A 15-day slow release of the compound of formula (Ib) from formulation IV was shown for all of the monkeys with the drug plasma concentrations in a range of about 40 ng/mL. In contrast, monkeys dosed with the compound of formula (I) in the absence of PLG had plasma concentrations that dropped to below detectable limits within one day.

### EXAMPLE 8

### In Vitro Drug Release of the Compound of Formula (Ia) from PLG (24 KD) Gels

(a) Formulation of 3% Compound of Formula (Ia) in 35% PLG (24KD) and NMP/TA (2:1)

The compound of formula (Ia) (26.7 mg) was added into a solution containing 0.3031 g of PLG (24KD, 50:50 polymer ratio) and 0.571 g of NMP/TA (2:1). The mixture was stirred at room temperature and resulted in a viscous liquid formulation which consisted of 2.96% compound of formula (Ia), 33.65% PLG, 42.21% NMP and 21.18% TA (w/w).

(b) Formulation of 5% Compound of Formula (Ia) in 35% PLG (24KD) and NMP/TA (4:1)

A solvent mixture of NMP/TA (4:1, w/w) was prepared from 4.012 g of NMP and 1.007 g of TA. A portion of the solvent mixture was treated with PLG (24KD, 50:50 polymer ratio, 0.3001 g). The resulting 35% PLG gel solution in MP/TA (4:1) was further stirred with 44.7 mg of the compound of formula (Ia) at room temperature and became a viscous liquid which consisted of 4.92% compound of formula (Ia), 33.15% PLG, 49.50% NMP and 12.43% TA (w/w).

### EXAMPLE 9

### In Vitro Drug Release of the Compound of Formula (Ia) from PLG (24KD) Gels

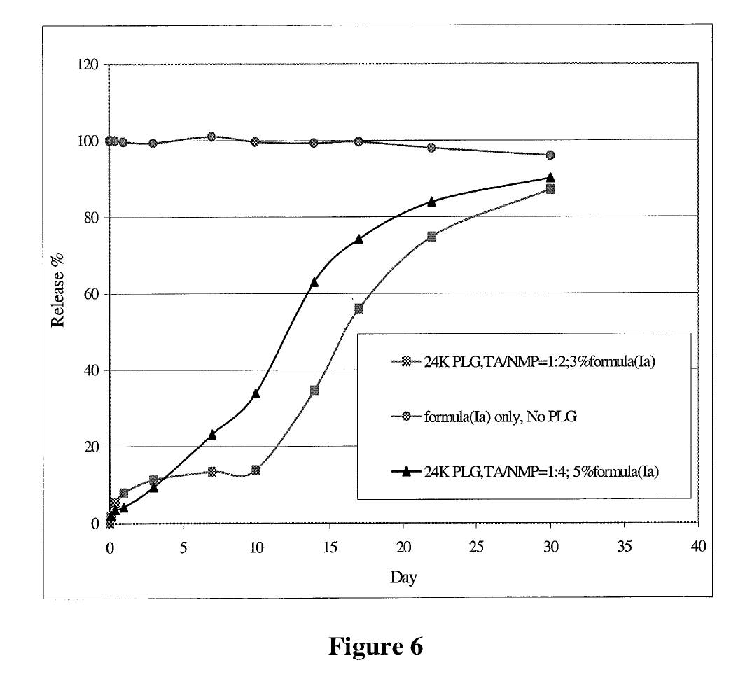

The in vitro drug release profiles of PLG (24 KD) gel formulations were obtained by the methods described in Example 3. As shown in FIG. 6, an increase of PLG molecular weight from 13KD to 24 KD significantly prolonged the in vitro release of the compound of formula (Ia) from the PLG gel, demonstrating sustained release for 30 days.

Using the procedures described in the preceding examples, PLG gel formulations can also be prepared for the following peptides:

N-Ac-DalloIle-Thr-Ser-Ile-Arg-ProNHCH₂CH₃;

N-Ac-Thr-Gln-Ile-Arg-ProNHCH₂CH₃ (SEQ ID NO:2);

N-Ac-DalloIle-Ser-Ser-Ile-Arg-ProNHCH₂CH₃;

N-Ac-Gly-Val-DalloIle-Ser-Gln-Ile-Arg-ProNHCH₂CH₃; and

N-Ac-Gly-Gln-DIle-Thr-Nva-Ile-Arg-Pro-DAlaNH₂.

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is

1. A pharmaceutical composition comprising:
(a) a therapeutically effective amount of a compound of formula (I) R¹-Xaa₁-Xaa₂-Xaa₃-Xaa₄-Xaa₅-Xaa₆-Ile-Arg-Pro-Xaa₁₀ (I), (SEQ ID NO:1);
wherein
R¹ is CH₃—C(O)—;
Xaa₁ is absent or sarcosyl;
Xaa₂ is absent or glycyl;
Xaa₃ is absent or selected from the group consisting of glutaminyl and valyl;
Xaa₄ is absent or selected from the group consisting of D-alloisoleucyl and D-isoleucyl;
Xaa₅ is selected from the group consisting of seryl and threonyl;
Xaa₆ is selected from the group consisting of glutaminyl, norvalyl, and seryl; and
Xaa₁₀ is selected from the group consisting of —NHCH₂CH₃ and D-alanylethylamide; provided that when Xaa₄ is D-alloisoleucyl, Xaa₁ is absent;
(b) poly(lactide-co-glycolide); and
(c) an organic solvent.

2. The pharmaceutical composition of claim 1 wherein the compound of formula (I) is selected from the group consisting of
N-Ac-Sar-Gly-Val-DIle-Thr-Nva-Ile-Arg-ProNHCH₂CH₃; and
N-Ac-Sar-Gly-Val-DIle-Thr-Gln-Ile-Arg-ProNHCH₂CH₃:

3. The pharmaceutical composition of claim 1 wherein the compound of formula (I) is selected from the group consisting of
N-Ac-DalloIle-Thr-Ser-Ile-Arg-ProNHCH₂CH₃;
N-Ac-Thr-Gln-Ile-Arg-ProNHCH₂CH₃ (SEQ ID NO:2);
N-Ac-DalloIle-Ser-Ser-Ile-Arg-ProNHCH₂CH₃;
N-Ac-Gly-Val-DalloIle-Ser-Gln-Ile-Arg-ProNHCH₂CH₃; and
N-Ac-Gly-Gln-DIle-Thr-Nva-Ile-Arg-Pro-DAlaNH₂.

4. The pharmaceutical composition of claim 1 which comprises between about 1% and about 15% (w/w) of the compound of formula (I).

5. The pharmaceutical composition of claim 1 which comprises between about 3% and about 5% (w/w) of the compound of formula (I).

6. The pharmaceutical composition of claim 1 which comprises between about 25% and about 45% (w/w) poly(lactide-co-glycolide).

7. The pharmaceutical composition of claim 6 which comprises about 35% (w/w) poly(lactide-co-glycolide).

8. The pharmaceutical composition of claim 1 wherein the poly(lactide-co-glycolide) has a weight of between about 6 and about 60 KD.

9. The pharmaceutical composition of claim 8 wherein the poly(lactide-co-glycolide) has a weight of between about 13 and about 24 KD.

10. The pharmaceutical composition of claim 1 wherein the organic solvent is N-methyl-2-pyrrolidinone.

11. The pharmaceutical composition of claim 1 wherein the organic solvent is triacetin.

12. The pharmaceutical composition of claim 1 wherein the organic solvent is a mixture of N-methyl-2-pyrrolidinone and triacetin.

13. The pharmaceutical composition of claim 12 wherein the N-methyl-2-pyrrolidinone and the triacetin are in a weight ratio of from about 1:2 to about 6:1.

14. The pharmaceutical composition of claim 13 wherein the N-methyl-2-pyrrolidinone and the triacetin are in a weight ratio of about 2:1.

15. The pharmaceutical composition of claim 13 wherein the N-methyl-2-pyrrolidinone and the triacetin are in a weight ratio of about 1:1.

16. A pharmaceutical composition comprising
(a) about 3% to about 5% (w/w) of the compound of formula (Ia) N-Ac-Sar-Gly-Val-DIle-Thr-Nva-Ile-Arg-ProNHCH₂CH₃ (Ia);
(b) about 35% (w/w) poly(lactide-co-glycolide); and
(c) about a 2:1 (w/w) mixture of N-methylpyrrolidinone and triacetin.

17. A pharmaceutical composition comprising
(a) about 3% (w/w) of the compound of formula (Ib) N-Ac-Sar-Gly-Val-DIle-Thr-Gln-Ile-Arg-ProNHCH₂CH₃ (Ib);
(b) about 35% (w/w) poly(lactide-co-glycolide); and
(c) about a 1:1 (w/w) mixture of N-methylpyrrolidinone and triacetin.

18. A method for preparing a pharmaceutical composition comprising:
(a) combining between about 25% and about 45% (w/w) poly(lactide-co-glycolide) and about 1% to about 15% (w/w) of a compound of formula (I) in an organic solvent; and
(b) stirring the product of step (a).

19. A method for preparing a pharmaceutical composition comprising:
(a) dissolving between about 25% and about 45% (w/w) poly(lactide-co-glycolide) in an organic solvent selected from the group consisting of N-methyl-2-pyrrolidinone, triacetin, 2-pyrrolidinone, and mixtures thereof;
(b) treating the product of step (a) with about 2% to about 10% (w/w) of a compound of formula (I); and
(c) stirring the product of step (b).

20. The method of claim 19 wherein the compound of formula (I) is selected from the group consisting of
N-Ac-Sar-Gly-Val-DIle-Thr-Nva-Ile-Arg-ProNHCH₂CH₃; and
N-Ac-Sar-Gly-Val-DIle-Thr-Gln-Ile-Arg-ProNHCH₂CH₃.

21. The method of claim 19 wherein the compound of formula (I) is selected from the group consisting of
N-Ac-DalloIle-Thr-Ser-Ile-Arg-ProNHCH₂CH₃;
N-Ac-Thr-Gln-Ile-Arg-ProNHCH₂CH₃ (SEQ ID NO:2);
N-Ac-DalloIle-Ser-Ser-Ile-Arg-ProNHCH₂CH₃;
N-Ac-Gly-Val-DalloIle-Ser-Gln-Ile-Arg-ProNHCH₂CH₃; and
N-Ac-Gly-Gln-DIle-Thr-Nva-Ile-Arg-Pro-DAlaNH₂.

22. The method of claim 19 wherein the pharmaceutical composition comprises about 35% (w/w) poly(lactide-co-glycolide).

23. The method of claim 19 wherein the poly(lactide-co-glycolide) has a weight of between about 13 and about 24 KD.

24. The method of claim 19 wherein the organic solvent is N-methyl-2-pyrrolidinone.

25. The method of claim 19 wherein the organic solvent is triacetin.

26. The method of claim 19 wherein the organic solvent is a mixture of N-methyl-2-pyrrolidinone and triacetin.

27. The method of claim 26 wherein the N-methyl-2-pyrrolidinone and the triacetin are in a weight ratio of from about 1:2 to about 6:1.

28. The method of claim 27 wherein the N-methyl-2-pyrrolidinone and the triacetin are in a weight ratio of from about 2:1 to about 1:1.

29. The method of claim 19 wherein step (c) is conducted at about 20° C. to about 25° C.

30. A method for preparing a pharmaceutical composition comprising:
(a) dissolving about 35% (w/w) 13 KD poly(lactide-co-glycolide) in about a 2:1 (w/w) mixture of N-methyl-2-pyrrolidinone and triacetin;
(b) treating the product of step (a) with about 3% to about 5% (w/w) of the compound of formula (Ia); and
(c) stirring the product of step (b) at about 20° C. to about 25° C.

31. A method for preparing a pharmaceutical composition comprising:
(a) dissolving about 35% (w/w) 13 KD poly(lactide-co-glycolide) in about a 1:1 (w/w) mixture of N-methyl-2-pyrrolidinone and triacetin;
(b) treating the product of step (a) with about 3% (w/w) of the compound of formula (Ib); and
(c) stirring the product of step (b) at about 20° C. to about 25° C.

32. A method for providing sustained delivery of a peptide comprising administering to a subject a pharmaceutical composition comprising
(a) about 1% to about 15% (w/w) of a compound of formula (I) R¹-Xaa₁-Xaa₂-Xaa₃-Xaa₄-Xaa₅-Xaa₆-Ile-Arg-Pro-Xaa₁₀ (I), (SEQ ID NO:1);
wherein
R¹is CH₃—C(O)—;
Xaa₁ is absent or sarcosyl;
Xaa₂ is absent or glycyl;
Xaa₃ is absent or selected from the group consisting of glutaminyl and valyl;
Xaa₄ is absent or selected from the group consisting of D-alloisoleucyl and D-isoleucyl;
Xaa₅ is selected from the group consisting of seryl and threonyl;
Xaa₆ is selected from the group consisting of glutaminyl, norvalyl, and seryl; and
Xaa₁₀ is selected from the group consisting of —NHCH₂CH₃ and D-alanylethylamide; provided that when Xaa₄ is D-alloisoleucyl, Xaa₁ is absent;
(b) about 25% to about 45% (w/w) poly(lactide-co-glycolide); and
(c) an organic solvent selected from the group consisting of N-methyl-2-pyrrolidinone, triacetin, and mixtures thereof.

33. The method of claim 32 wherein the compound of formula (I) is selected from the group consisting of
N-Ac-Sar-Gly-Val-DIle-Thr-Nva-Ile-Arg-ProNHCH₂CH₃; and
N-Ac-Sar-Gly-Val-DIle-Thr-Gln-Ile-Arg-ProNHCH₂CH₃.

34. The method of claim 32 wherein the compound of formula (I) is selected from the group consisting of
N-Ac-DalloIle-Thr-Ser-Ile-Arg-ProNHCH₂CH₃;
N-Ac-Thr-Gln-Ile-Arg-ProNHCH₂CH₃ (SEQ ID NO:2);
N-Ac-DallonIle-Ser-Ser-Ile-Arg-ProNHCH₂CH₃;
N-Ac-Gly-Val-DalloIle-Ser-Gln-Ile-Arg-ProNHCH₂CH₃; and
N-Ac-Gly-Gln-DIle-Thr-Nva-Ile-Arg-Pro-DAlaNH₂.
